(19) United States
(12) Patent Application Publication
(10) Pub. No.: US 2006/0018986 A1
(43) Pub. Date: Jan. 26, 2006

(54) Extracts of decaffeinated coffee beans and orally administrable compositions comprised thereof for stimulating the sebaceous function of the skin

(75) Inventor: Lionel Breton, Versailles (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignees: L'OREAL, PARIS (FR); NESTEC S.A., VEVEY (CH)

(21) Appl. No.: 11/150,163

(22) Filed: Jun. 13, 2005

Related U.S. Application Data
(63) Continuation of application No. PCT/EP03/15026, filed on Dec. 12, 2003.
(60) Provisional application No. 60/441,734, filed on Jan. 23, 2003.

(30) Foreign Application Priority Data
Dec. 13, 2002 (FR) 02/15867

(51) Int. Cl. A61K 36/185

(52) U.S. Cl. 424/776

(57) ABSTRACT

Orally administrable cosmetic/nutritional/pharmaceutical compositions suited for stimulating the sebaceous function of the skin, and/or for treating/preventing dry skin and/or skin aging, contain a thus effective amount of an extract of decaffeinated coffee beans, formulated into an orally administrable, physiologically acceptable medium therefor.

### CROSS-REFERENCE TO PRIORITY/PCT/PROVISIONAL APPLICATIONS

This application claims priority under 35 U.S.C. § 119 of FR-02/15867, filed Dec. 13, 2002, and of provisional application Ser. No. 60/441,734, filed Jan. 23, 2003, and is a continuation of PCT/EP 2003/015026, filed Dec. 12, 2003 and designating the United States (published in the English language on Jul. 1, 2004 as WO 2004/054535 A1); each hereby expressly incorporated by reference and each assigned to the assignee hereof.

### CROSS-REFERENCE TO COMPANION APPLICATION

Copending application Ser. No. ______ [Attorney Docket No. 010830-134], filed concurrently herewith and also assigned to the assignee hereof.

### BACKGROUND OF THE INVENTION

1. Technical Field of the Invention

The present invention relates to the formulation of extracts of decaffeinated coffee beans into compositions suited for oral administration to stimulate the sebaceous function of the skin, and in particular to correct the disorders associated with a dry skin.

The present invention relates in particular to cosmetic, nutritional or pharmaceutical compositions suited for administration by the oral route for the stimulation of the sebaceous function of the skin. This invention also relates to cosmetic procedures (regime or regimen) for the treatment of dry skins.

2. Description of Background and/or Related and/or Prior Art

An oligoseborrheic dry skin is characterized by an inadequate secretion and excretion of sebum. Conventionally, a concentration of sebum lower than 100 μg/cm² measured on the forehead is considered as characteristic of such a dry skin.

A dry skin is often associated with a desquamation deficiency, a dull complexion, an atonic skin texture. Micro-inflammatory symptoms, dermatitis in particular, appear more frequently in cases of dry skin.

Sensations of discomfort such as spasmodic twitches are usually experienced in the face by subjects having dry skin.

All of these disorders progress with age, since chronological aging is conventionally accompanied by loss of function of the sebaceous adnexa.

On the other hand, it is conventionally admitted that normally greasy skins exhibit an improved picture on aging compared with dry skins, This effect might be due to the fact that vitamin E is excreted by the sebaceous route (Thiele et al., J. Invest Dermatol., 1999; 113; 1006-10).

The sebum is the natural product of the sebaceous gland which constitutes an adnex of the pilosebaceous unit. Together with the sweat, produced by the eccrine or apocrine glands, it constitutes a natural hydrating agent of the epidermis.

The sebaceous secretion is under the control of different afferences of nervous origin. Cartlidge et al., (Br. J. Dermatol., 1972; 86(1), 61-63) have defined the modulatory role of the cholinergic system (para-lymphatic) system on seborrhea. It is known, moreover, that the dopaminergic system, when it is established, as is the case in the Parkinson syndrome, leads to hyperseborrhea which can be corrected by L-DOPA (J. C. Villares et al., Acta. Neurol. Scand., 80(1), 5Z-63). It is also known that the cholinergic system, through the intermediary of the muscarinic receptor subtype, antagonises the release of dopamine (Pharmacologie, M. Schorderét et al., p71, Ed. Frison-Roche, ISBN 2-05-100910-4).

An activation of the dopaminergic system and/or an inhibition of the cholinergic system (via the muscarinic receptors) might thus lead to a diminution of lipogenesis and/or excretion of sebum.

On the other hand, a limitation of the dopaminergic stimulation and/or an activation of the cholinergic system (via the muscarinic receptors) might lead to an increased secretion and/or production of sebum. A cholinomimetic activity of the muscarinic type has been found in alcoholic fractions of decaffeinated or undecaffeinated coffee beans (S. Y. Tse, J. Pharm. Sci., 1991, 80(7), 665-669 and S. Y. Tse, J. Pharm. Sci., 1992, 81(7), 449-452).

### SUMMARY OF THE INVENTION

It has now surprisingly and unexpectedly been determined that oral administration of a composition containing an extract of decaffeinated coffee beans has a beneficial effect on the stimulation of the sebaceous function of the skin.

Coffee trees are small trees with smooth-margined, perennial, coriaceous, glossy leaves (10-15×4-6 cm). The white, fragrant flowers are grouped in whorls at the axil of the leaves. The fruit is a green drupe, which becomes red at maturity and usually contains two planar-convex berries which are made contiguous through their planar face. Although only two species supply the essential needs of the coffee market (C. arabica and C. canephora), many species of coffee trees exist in the wild state in the tropical forests of East Africa.

The berry is oval (10-15×6-8 mm), convex on the dorsal face, flattened on the ventral face which is traversed by a longitudinal groove, the hilum. Hard and greenish, it is odorless. The microscopic examination of the green coffee powder reveals fusiform fibers derived from the tegument and cells of albumen: polyhedral, their wall is nacreous and irregularly thickened in a bead-like structure; they contain oily droplets.

The coffee “bean” is obtained by the moist route (fermentation, washing) or the dry route (drying, followed by mechanical decortication) starting from the coffee “cherry”, i.e., from the drupes. The reduction to pulp removes the red epicarp and the fleshy mesocarp; it leads to the coffee “husk”. It is after husking (removal of the lignified endocarp) that the coffee berry” (or bean) is obtained.

More than 50% of the dry matter of the green coffee berry is represented by carbohydrates, essentially polysaccharides. The proteins represent 10 to 12% of this mass, the lipids 10 to 18%. The unsaponiflable fraction of the crude lipids is considerable (more than 10%): in addition to sterols, hydrocarbons, tocopherols, diterpenic alcohols (cafestol, kahweol and kauranic derivatives) are observed to be present in the free state and, in particular, in the state of fatty acid esters. The coffee berry contains about 5% of phenolic acids: quinic acid, caffeic acid, chlorogenic acid. The caffeine content is variable: from 0.6 to 2% and more than 3% for certain canephora (robusta variety).

On torrefaction the texture and the composition of the berry change considerably. The water content is reduced, the berry swells, the polysaccharides are very degraded (forming in particular soluble products), pigments form (polycondensed furans) and the extremely complex flavor develops (several hundred compounds: alcohols, phenols, aldehydes, furanic and pyrrolic derivatives, hydrocarbons, thiophenes, etc.).

As far as the present inventor is aware, it has never been suggested that an extract of decaffeinated coffee beans be used in the preparation of a composition formulated for oral administration and intended for the stimulation of the sebaceous function of the skin, in particular for the treatment of dry skins.

Thus, the present invention features formulating an extract of decaffeinated coffee beans into compositions suited to stimulate the sebaceous function of the skin, such compositions being formulated for oral administration.

### DETAILED DESCRIPTION OF BEST MODE AND SPECIFIC/PREFERRED EMBODIMENTS OF THE INVENTION

In the text which follows “coffee beans” must be understood to mean the bean obtained by the moist route (fermentation, washing) or by the dry route (drying followed by mechanical husking) starting from the coffee “cherry”, after husking as described above.

“Extract” must be understood to mean all of the compounds obtained starting from an alcoholic or aqueous-alcoholic extraction of a crude product, in this instance decaffeinated coffee beans, roasted or unroasted.

The production of sebum by the skin can be determined by the measurement of the amount of sebum according to the standard so-called sebumetric procedure described, for example, in the L'Oréal patent FR-2-368,708 or FR-2-404,845.

By “stimulation of the sebaceous function of the skin” is meant a significant stimulation of the amount of sebum in the skin.

The species of coffee trees selected for the preparation of the extracts of coffee beans used in the compositions are advantageously selected from the Coffea species.

In a specific embodiment, the extract is derived from coffee beans selected from the species Coffea arabica, Coffea robusta, Coffea canephora or Coffea iberica. The extract may be obtained starting from roasted coffee beans. It can also be obtained from unroasted coffee beans.

For use according to the invention, the extract of coffee beans is decaffeinated.

In particular, a coffee bean extract can be obtained by an aqueous-alcoholic or alcoholic extraction of coffee beans, and preferably by an extraction with the aid of methanol, ethanol or propanol. Preferably, it does not contain the fractions of coffee beans extractable by non-polar solvents.

Methods for the preparation of decaffeinated coffee extracts are described in particular by S. Y. H. Tse (see above) and in the Examples presented hereafter.

The present invention also features cosmetic, nutritional or pharmaceutical compositions suitable for oral administration containing the extract of decaffeinated coffee beans, suited to stimulate the sebaceous function of the skin. In particular, the compositions according to the invention are suited for the treatment and/or the prevention of dry skins or skin aging. The proportion of decaffeinated coffee bean extract in the composition will of course be determined as a function of the desired effect on the stimulation of the sebaceous function of the skin and the mode of administration of the composition.

The compositions for administration by the oral route may be formulated in any galenical form suitable for this mode of administration, for example in the form of scored or unscored tablets, granules, capsules, gelatin capsules, solutions, suspensions or solutions containing an appropriate excipient.

The composition may be any food or pharmaceutical product, or a cosmetic product for oral application. Examples for food or pharmaceutical carriers are milk, yogurt, curd, cheese, fermented milks, milk based fermented products, ice-creams, fermented cereal based products, milk based powders, infant formulae or pet food, or tablets, liquid bacterial suspensions, dried oral supplement, wet oral supplement, dry tube-feeding or wet tube-feeding.

Preferably, a composition according to the invention is a nutritional supplement, presented in the form of a solid composition of the tablet, granule, capsule, gelatin capsule type and containing an extract of decaffeinated coffee beans as defined above and at least one adjuvant suitable for oral administration.

In this respect the adjuvants for oral compositions, in particular for dietary supplements, are known to the specialist. Mention may be made, among others and for purely illustrative purposes, of lubricants such as magnesium stearate, products for instantaneous solubilisation, gelling agents, thickeners, moisturizers, fatty and/or aqueous compounds, preservatives, texturizing, flavoring and/or coating agents, anti-oxidants and coloring materials usually used in foods.

The compositions according to the present invention may contain, in addition, lipids, polyphenols, taurine, probiotic microorganisms, vitamins and/or oligo-elements. If probiotics are used, they may be included in a live form, semi-active or in a desactivated form, e.g., as a lyophilized powder. Also, culture supernatants of the micro-organisms may be included in the composition. They may be selected from the group consisting of lactic acid bacteria, in particular Lactobacilli and/or Bifidobacteria and are more preferably selected among the group consisting of Lactobacillus johnsonii, Lactobacillus reuteri, Lactobacillus rhamnosus, Lactobacillus paracasei, Lactobacillus casei, Bifidobacterium bifidum, Bifidobacterium breve, Bifidobacterium animalis, Bifidobacterium infantis, Bifidobacterium dolescentis and Bifidobacterium pseudocatenulatum. According to a most preferred embodiment, the strains used are Lactobacillus johnsonii (La1) deposited on Jun. 30, 1992, under Budapest Treaty with the Institute Pasteur and receiving the deposit no. CNCM 1-1225 or Lactobacillus paracasei (ST11) deposited on Jan. 12, 1999, with the Institute Pasteur according to the Budapest Treaty and receiving the deposit no CNCM 1-2116. The following compounds may for example be used alone or in combination: zinc and its salts including zinc sulfate and zinc glucanate, the vitamins B5, B6, B8, C, E or PP, β-carotene and the carotenoids, extracts of garlic in particular in the form of allyl sulfide or oil garlic, selenium, curcumin, the curcuminoids, niacin, lithospermic acid and adenosine. It is understood that the specialist will select such active compounds and, where possible, combine them in such a manner as to improve the effects expected of the composition which is the object of the invention by preventing the desired activity of interest from being inhibited or attenuated.

The compositions suited for oral administration contain an extract of decaffeinated coffee beans in a quantity ranging from 0.1% to 80% by weight of the composition and preferably from 1% to 50% by weight of the composition.

Chlorogenic acid which is a phenolic compound naturally present in some coffee bean extracts, is not involved in the treatment of dry skins.

Chlorogenic acid is thus not an active agent of the compositions for the treatment and/or the prevention of dry skins according to the invention.

Accordingly, in a specific embodiment, chlorogenic acid is present in the composition containing decaffeinated coffee beans in a quantity inferior or equal to 0.1% by weight of the composition.

The present invention also features a cosmetic procedure for the prevention and/or the treatment of dry skins, or a procedure for the prevention and/or the cosmetic treatment of skin aging, which entails administering by the oral route a composition containing an extract of coffee beans, such as described above.

The daily doses of decaffeinated coffee bean extract administered by the oral route for the treatment of dry skins may preferably be comprised between 0.01 and 5,000 mg/day. Preferentially, the coffee bean extract is present in the composition according to the invention in a quantity permitting its administration at a dose comprised between 0.5 and 1,000 mg/day.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

### Example 1

### Preparation of a Roasted Extract of Coffea robusta

0.5 kg of roasted coffee beans is reduced to a powder by grinding with the Turrax apparatus at 24,000 rev/min for 1 minute at 4° C. (ice bath).

The powder obtained is mixed with 5 liters of 0.05M phosphate buffer at pH 8.5. The entire mixture is stirred for 30 minutes at 4° C., then centrifuged at 10,000 G at 4° C. The supernatant is filtered through a 0.22 μm filter (sterilizing filtration).

The extract is then fractionated by ultrafiltration through a Sartorius type membrane in order to remove from it oxidation phenomena.

The extract is then lyophilized. 29.5 grams of active extract called “lyophilized extract” are thus obtained.

Caffeine is then removed by supercritical chromatography (CO₂ is used as carrier gas). 25.5 grams of active extract called “decaffeinated lyophilized extract” are thus obtained.

### Example 2

### Examples of Formulations Illustrating the Invention and in Particular the Compositions According to the Invention

These compositions were obtained by the simple mixing of the different constituents.

Composition 1: Soft capsules:

| Excipients: | | |
|---|---|---|
| Soya oil | 40 | mg |
| Wheat germ oil | 85 | mg |
| Soya lecithins | 25 | mg |
| Vitamins: | | |
| Natural tocopherols | 3 | mg |
| Vitamin C palmitate | 150 | mg |
| Constituents: | | |
| Decaffeinated lyophilized extract of Coffea robusta | 15 | mg |
| Borage oil | 200 | mg |
| Blackcurrant pip oil | 150 | mg |

Composition 2: Soft capsules:

| Excipients: | | |
|---|---|---|
| Soya oil | 40 | mg |
| Wheat germ oil | 85 | mg |
| Soya lecithins | 25 | mg |
| Vitamins: | | |
| Natural tocopherols | 3 | mg |
| Constituents: | | |
| Decaffeinated lyophilized extract of Coffea robusta | 150 | mg |
| Borage oil | 200 | mg |
| Evening primrose oil | 200 | mg |

Composition 3: Soft capsules:

| Excipients: | | |
|---|---|---|
| Soya oil | 40 | mg |
| Wheat germ oil | 85 | mg |
| Soya lecithins | 25 | mg |
| Vitamins: | | |
| Natural tocopherols | 3 | mg |
| Constituents: | | |
| Decaffeinated lyophilized extract of Coffee robusta | 50 | mg |
| Borage oil | 200 | mg |
| Evening primrose oil | 200 | mg |
| Lyophilized Lactobacillus | 200 | mg |

Composition 4: Soft capsules:

| Excipients: | | |
|---|---|---|
| Soya oil | 40 | mg |
| Wheat germ oil | 5 | mg |
| Soya lecithins | 25 | mg |
| Vitamins: | | |
| Natural tocopherols | 3 | mg |
| Constituents: | | |
| Decaffeinated lyophilized extract of Coffea robusta | 150 | mg |
| Borage oil | 200 | mg |
| Evening primrose oil | 200 | mg |
| Vitamin C | 50 | mg |
| Calcium glucanate | 200 | mg |
| Magnesium stearate | 400 | mg |
| Lyophilized Lactobacillus sp | 300 | mg |

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. An orally administrable cosmetic/nutritional/pharmaceutical composition suited for stimulating the sebaceous function of the skin, comprising a thus effective amount of an extract of decaffeinated coffee beans, formulated into an orally administrable, physiologically acceptable medium therefor.

2. The orally administrable composition as defined by claim 1, said extract being of decaffeinated coffee beans of the species Coffea.

3. The orally administrable composition as defined by claim 2, said extract being of decaffeinated coffee beans of the species Coffea arabica, Coffea robusta, Coffea canephora or Coffea iberica.

4. The orally administrable composition as defined by claim 1, said decaffeinated coffee beans having been roasted.

5. The orally administrable composition as defined by claim 1, said decaffeinated coffee beans being unroasted.

6. The orally administrable composition as defined by claim 1, said extract of decaffeinated coffee beans comprising from 0.1% to 80% by weight thereof.

7. The orally administrable composition as defined by claim 1, said extract of decaffeinated coffee beans comprising from 1% to 50% by weight thereof.

8. The orally administrable composition as defined by claim 1, said extract having been obtained by aqueous-alcoholic or alcoholic extraction.

9. The orally administrable composition as defined by claim 1, comprising no greater than 0.1% by weight of chlorogenic acid.

10. The orally administrable composition as defined by claim 1, formulated as a solid and comprising at least one orally administrable adjuvant.

11. The orally administrable composition as defined by claim 1, formulated as tablets, granules, capsules, gelatin capsules, a solution or a suspension.

12. The orally administrable composition as defined by claim 1, comprising a food or pharmaceutical carrier.

13. The orally administrable composition as defined by claim 1, further comprising a lipid, polyphenol, taurine, probiotic microorganism, vitamin and/or oligo-element.

14. A regime or regimen for stimulating the sebaceous function of the skin, comprising orally administering to an individual in need of such treatment, an orally administrable cosmetic/nutritional/pharmaceutical composition comprising a thus effective amount of an extract of decaffeinated coffee beans, formulated into an orally administrable, physiologically acceptable medium therefor.

15. A regime or regimen for the treatment and/or prevention of dry skin or for the treatment and/or prevention of skin aging, comprising orally administering to an individual in need of such treatment, an orally administrable cosmetic/nutritional/pharmaceutical composition comprising a thus effective amount of an extract of decaffeinated coffee beans, formulated into an orally administrable, physiologically acceptable medium therefor.

16. The regime or regimen as defined by claim 14, said extract being of decaffeinated coffee beans of the species Coffea arabica, Coffea robusta, Coffea canephora or Coffea iberica.

17. The regime or regimen as defined by claim 14, said decaffeinated coffee beans being unroasted.

18. The regime or regimen as defined by claim 15, said extract being of decaffeinated coffee beans of the species Coffea arabica, Coffea robusta, Coffea canephora or Coffea iberica.

19. The regime or regimen as defined by claim 15, said extract being of decaffeinated coffee beans being unroasted.
